(19) United States
(12) Patent Application Publication
(10) Pub. No.: US 2005/0096644 A1
(43) Pub. Date: May 5, 2005

(54) Energy delivery optimization for RF duty cycle for lesion creation

(76) Inventors: Jeffrey A. Hall, Birmingham, AL (US); Mudit Jain, Woodbury, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/698,026

(22) Filed: Oct. 30, 2003

(51) Int. Cl. A61B 18/14

(52) U.S. Cl. 606/41; 606/34

(57) ABSTRACT

A method of RF ablation includes choosing a duty cycle for an RF energy treatment depending on one or more static thermal properties of an electrode, and delivering the RF energy treatment to a tissue from the electrode. The method can include determining a thermal decay proximate the electrode, and changing a duty cycle of the RF energy depending on the thermal decay.

### FIELD OF THE INVENTION

This invention relates to the field of medical technology, and more specifically to a method and apparatus of delivering RF energy to tissue.

### BACKGROUND

Physicians employ radiofrequency (RF) ablation to treat various conditions, such as cardiac rhythm dysfunction. One technique for performing RF ablation is to insert a catheter having one or more electrodes into a body and against the tissue to be ablated. As RF energy passes through the catheter, the energy heats up and burns the tissue, creating a lesion. The lesion and subsequent scar tissue, which do not conduct electrical impulses, essentially form roadblocks for abnormal impulses responsible for the heart condition. This can restore the correct operation of the heart.

During RF ablation, adjacent endocardial tissue heats the electrodes via heat conduction through the tissue. Since portions of the electrodes are in contact with the blood, it is possible for clotting and boiling of blood to occur if those electrodes reach an excessive temperature. An electrode temperature above 100 degrees C. results in coagulum formation that coats the electrode, causing a rise in impedance that limits current flow and prevents further lesion expansion. This can result in a lesion that is not deep enough to prevent the abnormal signal irregularities. When these conditions arise, the ablation procedure must be stopped and the catheter removed and cleaned or replaced before the procedure can continue.

### SUMMARY

One aspect of the present system includes a catheter having an electrode and a temperature sensor, an RF energy source connected to the electrode for delivering RF energy via the electrode, and a controller for controlling a duty cycle of the RF energy. The controller is coupled to the temperature sensor and is adapted to change the duty cycle of the RF energy as a function of a thermal decay as determined by a measurement of change of temperature, as measured by the temperature sensor, over a time period.

One aspect provides a method of RF ablation including delivering an RF energy to a tissue from an electrode, determining a thermal decay over time proximate the electrode, and changing a duty cycle of the RF energy in response to the thermal decay.

### BRIEF DESCRIPTION OF THE DRAWINGS

FIG. 1 shows a schematic illustration of an RF ablation system according to one embodiment.

FIG. 2 shows a schematic illustration of an RF ablation system according to one embodiment.

FIG. 3 depicts a flowchart of a method of RF ablation according to one embodiment.

FIG. 4 shows a schematic illustration of an RF ablation system according to one embodiment.

FIG. 5 shows a flowchart of a method of RF ablation according to one embodiment.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

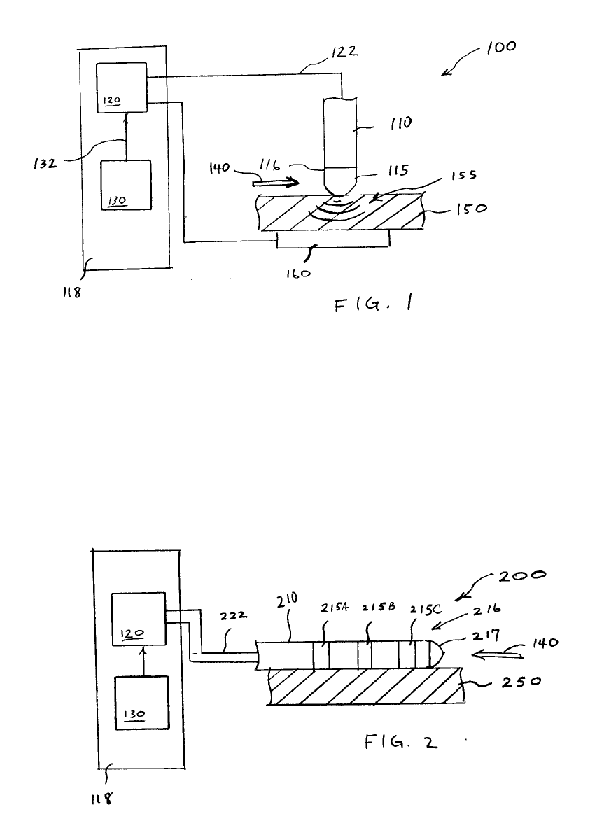

FIG. 1 shows a schematic illustration of an RF ablation system 100 according to one embodiment. System 100 includes a catheter 110 having an electrode 115 proximate a distal end 116, and a power system 118 including an RF energy source 120, and a controller 130. Catheter 110 is positioned such that electrode 115 is abutting and in contact with a tissue 150. For example, the electrode can be positioned at an endocardial surface to ablate a myocardial tissue. RF source 120 delivers energy 155 via electrode 115 to the tissue to create a lesion. The energy delivery is controlled by controller 130.

Catheter 110 includes a elongated body which extends from a proximal end to distal end 116. In one example, catheter 110 includes a steerable electrophysiological (EP) catheter capable of being percutaneously introduced into a biological site such as the atrium or ventricle of a heart. Catheter 110 is connected to RF source 120 by one or more connection wires 122 running up the elongated body from the proximal end to distal end 116. Electrode 115 is coupled to connection wire 122 to deliver the energy.

Electrode 115 includes an electrically conductive material and is part of the RF delivery assembly. In one embodiment, electrode 115 is a 5 mm (0.20″) sized tip electrode having a semi-spherical dome shape and dimensions of approximately 0.094″ diameter, 5 mm (0.20″) length and a band thickness of 0.0025 inches. In other examples, the electrode can have tip lengths ranging from 3-10 mm and thickness ranging from 0.0010 to 0.0050 inches. In one example, electrode 115 is formed of pure platinum, with a thermal constant of 0.24 seconds for the described 5 mm tip in an empirical flow bench measurement. In this example, the platinum tips would have a mass density of 21.45 grams/cc (0.77 lbs/cu in) and a thermal conductivity of 0.165 cal/cc×cm×s×° C. Other materials that can be used for electrode 115 include gold or platinum iridium. In one embodiment, electrode 115 is used to deliver RF energy to the tissue to form spot lesions.

In one example, a single electrode 115 is provided, to provide a uni-polar RF system. In a uni-polar system, a large surface area electrode 160 is placed externally to serve as a return electrode. In some embodiments, as will be discussed below, two or more electrodes can be located on the catheter to allow a bipolar RF system. In the bipolar method, the electrodes are oppositely charged and thus complete an electrical circuit between themselves. RF ablation systems using both uni-polar and bipolar at the same time are also within the scope of one or more embodiments of the present system. For example, in one embodiment both bipolar and unipolar energy delivery can be done concurrently and the power control system can control the phase angle of the power so that energy flows as desired between the electrodes and between the electrodes and the backplate electrode.

RF source 120 includes an RF generator incorporated within RF source 120. In one embodiment, RF source 120 includes a 9401 model, manufactured by Guidant, Inc. In various embodiments, the present technique of using RF energy for ablation of cardiac arrhythmias utilizes sine waves in the range of 100 to 750 kHz. In some embodiments, the generator outputs emissions of 500 kHz to a frequency of 750 kHz. In some embodiments, a frequency of 540 kHz is utilized.

RF lesion formation can be described as follows: with the onset of current flow, the temperature at the electrode 115—tissue 150 interface increases. This temperature increases until the energy applied via ablation catheter 110 equals that lost through conduction and convection of heat away from the site. One way energy is lost includes blood flow 140 moving past the electrode/tissue interface and over electrode 115. Once this steady-state temperature gradient has been achieved, there is no further lesion expansion. The total delivered energy can be calculated to be the product of power and duration.

As noted above, an electrode temperature above 100° C. results in coagulum formation that coats electrode 115, causing a rise in impedance that limits current flow and prevents further lesion expansion. This can result in a lesion that is not deep enough in tissue 150 to prevent the abnormal signal irregularities.

System 100 controls a duty cycle of the RF energy to provide for more efficient energy transfer, longer heating times, and larger lesion formation.

Duty cycle is a term defined as the proportion of time during which a component, device, or system is operated. For example, in the present system duty cycle is the ratio of time during which power is applied to the target tissue over the total time duration of the treatment, expressed as a percentage. For example, a power output having a duty cycle of 25% applied over a time duration of sixty seconds, the total on-load period during which power is actually applied would be fifteen seconds.

Controller 130 controls the RF energy output from RF source 120. This allows the RF energy to be applied so as to optimize the lesion formation. For example, in one embodiment, a duty cycle of the RF energy is controlled or set by controller 130. The controller provides a duty cycle signal over connector 132 to the power system to control the duty cycle. To allow for greater cooling by the blood flow 140, for example, the duty cycle is changed to provide a longer off-load period. The intent of this technique is to maximize the difference between the endocardial surface temperature and the deep tissue temperature several millimeters below the surface.

One influence which affects the temperature at the tissue surface is the amount of blood flow 140 over electrode 115 and over the tissue surface. Another factor is the thermal mass of electrode 115, which acts as a heat sink to cool the surface temperature by dissipation of heat through conduction. The thermal decay will be greater at the surface due to conduction of heat into the blood pool and the removal of heat by blood flow. By adjusting the duty cycle, the difference between the surface temperature and the deep tissue temperature can be maximized.

A decreased duty cycle will help increase the time available for cooling the tissue and the electrode surface in between energy applications. This will help reduce the time for coagulation and tissue charring because the tissue and the blood will be allowed to cool and not reach excessive temperatures.

Also, a decreased duty cycle will help produce deeper and larger lesions as one will be able to delivery energy for a longer net duration without reaching a thermal steady state.

FIG. 2 shows a schematic illustration of an RF ablation system 200 according to one embodiment. System 200 includes a catheter 210 having three band or ring electrodes 215A-215C and a tip electrode 217 arranged in a substantially linear array along a distal segment 216 of catheter 210. More or fewer ring electrodes can be provided in the electrode array configuration. System 200 also includes a power system 118 including an RF source 120 and a controller 130, such as described above in FIG. 1.

Catheter 210 is positioned such that electrodes 215A-215C are abutting and in contact with a tissue 250 in a side-fire configuration. RF source 120 delivers energy via one or more of electrodes 215A-215C or 217 to the tissue to create a lesion. The energy delivery is controlled by controller 130.

Catheter 210 includes a elongated body with one or more connection wires 222 running up the elongated body from the proximal end to distal end 113. Electrodes 215A-215C and 217 are coupled to the one or more connection wires 222 to deliver the energy delivered up the catheter.

Each of ring electrodes 215A-215C includes an electrically conductive material. In one embodiment, each electrode 215A-215C is a 3 mm ring electrode having an annular shape with an outside diameter of 0.0945 inches and a thickness of 0.0045 inches. In other examples, the electrode can have a length ranging from 2 mm to 6 mm and thickness ranging from 0.0025 to 0.0080 inches. In one example, electrodes 215A-215C are formed of a platinum material, with a thermal time constant of 0.24 sec. System 200 can be used as a bipolar or unipolar system depending on use. For example, in one example, a backplate electrode can be used as shown above for system 100.

Again, controller 130 controls the RF energy output from RF source 120. This allows the RF energy to be applied so as to optimize the lesion formation with this electrode configuration. For example, in one embodiment, a duty cycle of the RF energy is controlled or set by controller 130. In one example, controller 130 is adapted to change the duty cycle as a function of one or more thermal properties of electrodes 215A-215C, or 217.

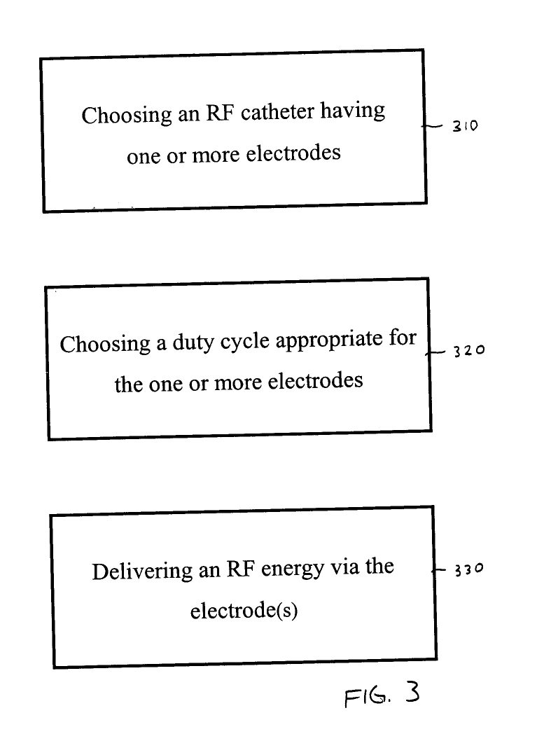

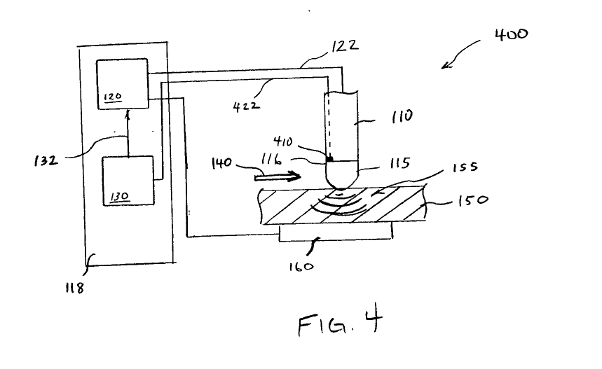

FIG. 3 shows a method of RF ablation in accordance with one embodiment. The method includes choosing an RF catheter having one or more electrodes (310), choosing a duty cycle which is appropriate for the one or more electrodes (320), and delivering an RF energy via the one or more electrode to a tissue (330).

In one example, choosing a duty cycle which is appropriate for a given electrode includes choosing a duty cycle which depends on one or more static or inherent thermal properties of the electrode. In various embodiments, the one or more thermal properties of the electrode used to determine the duty cycle can include one or more of a thermal constant of the electrode, a mass of the electrode, a surface area of the electrode, a material of the electrode, and a shape of the electrode. Again, duty cycle is a ratio defined by the ON portion versus the total period of an RF pulse.

Table 1 shows values for some example electrodes made of platinum with a thermal conductivity of 0.165 cal/cc×cm×s×° C. Tips 1, 2, and 3 are platinum tip electrodes having an empirical thermal time constant of approximately 240 ms, approximately 5 mm length, and a diameter of approximately 0.094 inches. Ring 1 and ring 2 are platinum ring electrodes having an empirical thermal constant of approximately 100 ms, a length of approximately 3 mm with a thickness of approximately 0.0045 inches and an outer diameter of approximately 0.0945 inches.

TABLE 1

| | | | peak power | |
|---|---|---|---|---|
| Electrode | Duty cycle | Period | (effective) | Flow rate |
| Tip 1 | 80-100% | >240 ms | 150 watts | 3 liters/min |
| | | | | (high flow) |
| Tip 2 | 80-100% | 120 ms | 150 watts | 3 liters/min |
| Tip 3 | 50% | 120 ms | 75 watts | 1 liter/min (low |
| | | | | flow) |
| Ring 1 | 10-20% | >100 ms | 150 watts | |
| Ring 2 | 1-10% | <100 ms | 150 watts | |

These are examples shown for specific electrodes. The present system applies to almost any electrode for RF ablation. In the examples for the tip electrodes, if the blood flow rate is high, the duty cycle can be between 80-100% even if the period is less than the thermal constant measured for a given electrode. If the blood flow is low, the duty cycle is lowered (to 50%, for example), to allow more time for cooling. When the period is greater than the time constant, more time is available for cooling and the duty cycle is raised to between 80%-100%. Similarly, for the ring electrodes, a longer period allows for a duty cycle of approximately 10%-20% and a shorter period allows for a duty cycle of 1%-10%. Again, the thermal decay caused by the given blood flow rate can allow for a higher duty cycle for a given period. More details on thermal decay and measurement of thermal decay using temperature measurements at the electrode will be discussed below.

In general, various embodiments can have RF pulses having pulse widths of approximately 0.01 ms-4 ms, and duty cycles of 1% to 100%, with the duty cycle being lowered when the pulse width is longer (or the period is shorter) such that the thermal time constant of the electrode governs the chosen duty cycle and thus the rate of energy delivery. This allows for more efficient energy transfer, longer heating times, and larger lesion formation.

Again, referring to Table 1, in one example of the present RF system, the system can utilize a platinum tip electrode having an empirical thermal time constant of approximately 240 ms, an area of approximately 5 mm and approximately 0.094 inches diameter. The RF energy can have an RF frequency of 500-540 kHz and a duty cycle of 50-100%.

In the examples shown above in Table 1, the longer duty cycle with longer time available for electrode/tissue cooling helps reduce the incidences of blood coagulation and tissue charring because both the tissue and the blood will be allowed to cool and not reach excessive temperatures. (For example, 100 degrees C.). Moreover, the different duty cycles help decrease surface disruption. Moreover, the present system optimizes the energy delivery to produce deeper and larger lesions by energy delivery for a longer net duration without reaching a thermal steady state.

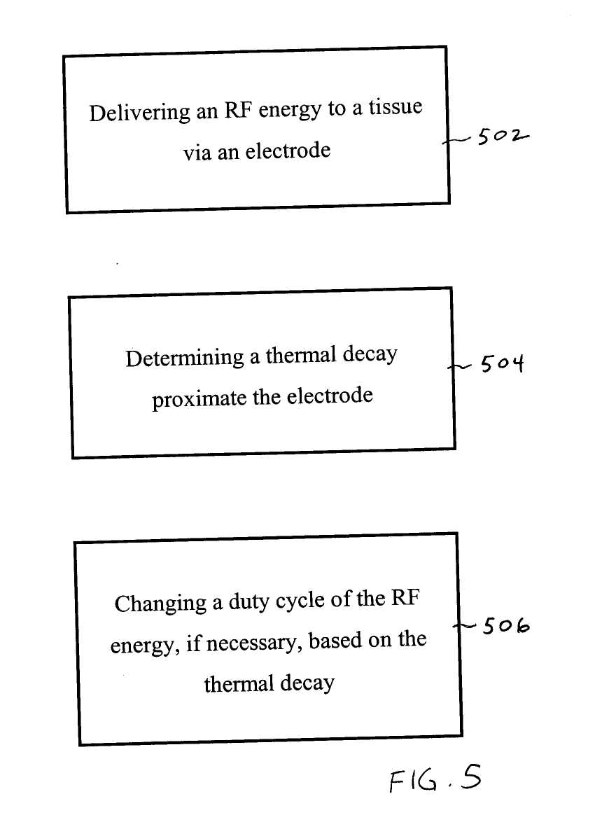

FIG. 4 shows a schematic illustration of an RF ablation system 400 according to one embodiment. In this example, system 400 includes some of the same members as discussed above for system 100, including catheter 110, electrode 115, and so on. Detailed description of these members will not be discussed for sake of clarity. It is also noted that one or more features of the following discussion also apply to RF ablation system 200 of FIG. 2, and the following discussion is applicable to one or more embodiments of that system also.

In this example, catheter 110 includes a temperature sensor 410 which is coupled to controller 130 by a connection 422. In one embodiment, controller 130 is adapted to change the duty cycle of the RF energy as a function of thermal decay proximate electrode 115. The thermal decay is dependent in part on the thermal properties of the electrode and on the location of the electrode due to blood flow past the electrode. By sensing the temperature change over time using temperature sensor 410, the thermal decay can be determined. For example, controller 130 can include a timer to sense a temperature change over time. Temperature sensor 410, such as a thermocouple, senses the temperature and sends a temperature signal to controller 130. The controller registers two or more of these temperature signals over a given time period. The temperature change over the time period allows controller 130 to calculate the thermal decay at the distal end of the catheter. The blood flow rate can then be estimated and the duty cycle or period changed as in Table 1, for example.

FIG. 5 shows a flowchart depicting a method of RF ablation in accordance with one embodiment. The method includes delivering an RF energy to a tissue via an electrode (502), determining a thermal decay proximate the electrode (504), and changing a duty cycle of the RF energy, if necessary, based on the thermal decay (506). By using the thermal decay as a guide, the controller can modify the duty cycle of the RF energy to help optimize the shape, size, continuity, and depth of the lesion. Again, since the thermal decay is dependent on blood flow, electrode contact, and thermal properties of the electrode, by monitoring the thermal decay at the temperature sensor, a value can be determined for blood flow and contact which can then be used as a factor to provide the optimum duty cycle. In one example, the controller estimates the blood flow by measuring the thermal decay over a period of time. For a given thermal mass of an electrode this is repeatable and consistent.

In general, the present system provides for choosing the duty cycle of an RF ablation treatment by keying the duty cycle to the thermal properties of the electrode. The duty cycle can be chosen based on catheter identification and knowing the thermal response time of the electrode. By basing the duty cycle on the electrode, the tissue temperature can be driven hotter and deeper than at the surface. Linear lesions can be more uniform and continuous. Moreover, by additionally monitoring the thermal decay at the thermocouple, a value can be obtained for blood flow and electrode contact to further optimize the duty cycle. This allows for the system to deliver energy for a longer net duration with deeper lesions because more power is delivered without surface disruption or reaching a thermal steady state.

Among other applications, the present system can be used to perform RF ablation to treat various cardiac rhythm dysfunctions such as ventricular tachycardia and atrial fibrillation. In one example, the method includes positioning the electrode at an endocardial surface to ablate myocardial tissue.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. A system for RF ablation comprising:
a catheter having an electrode and a temperature sensor;
an RF energy source connected to the electrode for delivering RF energy via the electrode; and
a controller for controlling a duty cycle of the RF energy, wherein the controller is coupled to the temperature sensor and is adapted to change the duty cycle of the RF energy as a function of a thermal decay as determined by a measurement of change of temperature, as measured by the temperature sensor, over a time period.

2. The system of claim 1, wherein the controller determines the thermal decay in part as a function of one or more static thermal properties of the electrode.

3. The system of claim 2, wherein one or more static thermal properties of an electrode include a thermal constant of the electrode.

4. The system of claim 2, wherein one or more thermal properties includes a mass of the electrode.

5. The system of claim 2, wherein one or more thermal properties includes surface area of the electrode.

6. The system of claim 1, wherein the duty cycle chosen can range from 1% to 100%.

7. The system of claim 1, wherein the duty cycle chosen can range from 1% to 20%.

8. The system of claim 1, wherein the duty cycle chosen can range from 80% to 100%.

9. The system of claim 2, wherein the one or more static thermal properties of the electrode include one or more of mass of the electrode, shape of the electrode, and thermal constant of the electrode.

10. The system of claim 1, wherein the electrode includes a tip electrode.

11. The system of claim 1, wherein the electrode includes a ring electrode.

12. The system of claim 1, wherein the electrode includes an approximately 5 mm tip with a diameter of approximately 0.094 inches.

13. The system of claim 1, wherein the electrode includes a ring electrode.

14. The system of claim 1, wherein the electrode includes an array of ring electrodes.

15. The system of claim 1, wherein the RF energy source delivers RF energy having a frequency of 500-750 kHz.

16. A system for delivering RF energy to an endocardial tissue, the system comprising:
a catheter having one or more electrodes proximate a distal end of the catheter, the catheter adapted for being positioned such that the one or more electrodes are adjacent the endocardial tissue; and
a power control system to provide power to the one or more electrodes, the power having a plurality of alternating on portions and off portions, one set of adjacent on and off portions defining a duty cycle;
wherein the on portions and off portions of the duty cycle are chosen as a function of thermal decay at the electrode and depending on one or more static thermal properties of the one or more electrodes.

17. The system of claim 16, wherein one or more static thermal properties of an electrode include a thermal constant of the electrode.

18. The system of claim 16, wherein one or more thermal properties includes a mass of the electrode.

19. The system of claim 16, wherein one or more thermal properties includes surface area of the electrode.

20. The system of claim 16, wherein the one or more static thermal properties of the electrode include one or more of mass of the electrode, shape of the electrode, and thermal constant of the electrode.

21. The system of claim 16, wherein one of the one or more electrode includes a tip electrode.

22. The system of claim 16, wherein one of the one or more electrodes includes a ring electrode.

23. A method of RF ablation comprising:
delivering RF energy to a tissue from an electrode;
determining a thermal decay over time proximate the electrode; and
changing a duty cycle of the RF energy in response to the thermal decay.

24. The method of claim 23, wherein determining a thermal decay includes measuring a temperature proximate the electrode at a first time and at a second later time.

25. The method of claim 23, wherein determining a thermal decay includes determining one or more thermal properties of the electrode.

26. A method of delivering RF energy to an endocardial tissue, the method comprising;
positioning a catheter having one or more electrodes such that at least one of the one or more electrodes is adjacent the endocardial tissue;
providing power to the one or more electrodes, the power having a duty cycle with an on portion and an off portion; and
measuring a temperature proximate the one or more electrodes at two different times, and modifying the duty cycle as a function of a thermal decay as determined by the temperatures measured.

27. The method of claim 26, further including modifying the duty cycle depending on one or more static thermal properties of the one or more electrodes.

28. The method of claim 27, wherein modifying the duty cycle depending on one or more static thermal properties includes modifying the duty cycle depending on a thermal constant of the one or more electrodes.
